(19) United States
(12) Patent Application Publication
(10) Pub. No.: US 2012/0051953 A1
(43) Pub. Date: Mar. 1, 2012

(54) Energy management for conversion of methanol into gasoline and methanol into olefins

(75) Inventor: Dennis J. O'Rear, Penngrove, CA (US)

(73) Assignee: Chevron U.S.A. In.

(21) Appl. No.: 12/807,065

(22) Filed: Aug. 25, 2010

(51) Int. Cl. F04B 47/08; C07C 27/00; C07C 1/00

(52) U.S. Cl. 417/375; 585/311; 518/702

(57) ABSTRACT

A process is disclosed for the conversion of a hydrocarbonaceous assets into gasoline. The hydrocarbonaceous assets are converted at a first methanol production facility into methanol by processes comprising gasification and methanol synthesis. At least a portion of the methanol is shipped to a second gasoline production facility that is at least 10 miles in distance from the first location. At the second gasoline production facility, at least a portion of the methanol is converted by a MTG process to produce a gasoline and LPG components and steam. At least a portion of the methanol may also be used in a methanol to olefin process to produce olefins and steam. The olefins may be alkylated with olefins to produce alkylates which may mixed with the gasoline. The steam from one or both of the MTG or MTO processes may also be used by processes such as driving pumps to unload the methanol, driving pumps to deliver the gasoline product, driving compressors to liquefy the LPG components, driving generators to produce electricity, heating other substances, to gasifying imported LNG, and combinations thereof.

### FIELD

The present disclosure generally to methods for making gasoline by integration of methanol to gasoline (MTG) and methanol to olefins (MTO) processes.

### DESCRIPTION OF THE RELATED ART

The developed and developing world markets need supplies of transportation fuels. Gasoline is one of the major fuels in use today and the near future. Petroleum is often found at sites remote from market sites. Petroleum can be readily transported to refineries near the markets where it is refined into gasoline.

In contrast, other hydrocarbonaceous assets are more difficult to transport as they are viscous or dense liquids, gases or solids. Examples of such hydrocarbonaceous assets include natural gas, coal, and plant based feedstocks such as wood, corn, switch grass, etc. These hydrocarbonaceous assets can be converted into methanol by various well know processes, including gasification, to form synthesis gas followed by methanol synthesis. Non-limiting examples well known to those skilled in the art for converting hydrocarbonaneous feedstocks into synthesis gas for conversion to methanol include steam methane reforming, partial oxidation, gasification, combined reforming and autoreforming.

The methanol can then be readily shipped to sites near the market sites where it can be converted into gasoline by a Methanol to Gasoline (MTG) process. Unfortunately, the MTG process produces significant quantities of light paraffins. These light paraffins are rich in non-quaternary isoparaffins, but these isoparaffins have low value because their high volatility makes blending into gasoline difficult or impossible. These isoparaffins then must be used in lower value applications such as fuel, and where possible, liquefied petroleum gas (LPG). Isopentane is particularly difficult to use because it cannot be used in either gasoline or in LPG.

Methanol to Olefins (MTO) is another process that converts methanol to a useful product, light olefins (ethylene, propylene and butenes). Ethylene is the olefin currently in geatest demand, and having the highest value. The propylene and butenes have lower value and improved uses for these olefins are desired.

There is a need for a process wherein otherwise hard to transport carbonaceous assets are converted to methanol. Then, the methanol is received at a gasoline manufacturing facility wherein the methanol can be efficiently converted into gasoline products.

In the current practice, the conversion of hydrocarbonaceous assets into gasoline at remote locations followed by shipping the gasoline to markets does not fully utilize the energy in the hydrocarbonaceous asset. This lost energy is equivalent to poor carbon management throughout the product value chain and increased greenhouse gases.

It is desirable to convert hydrocarbonaceous assets that are in locations remote from market into transportation fuels for use in developed markets. In the 1970's Mobil commercialized a technology in New Zealand which converted natural gas into methanol, and then converted the methanol into gasoline by a Methanol-to-Gasoline process (MTG). However recent concerns over energy management and greenhouse gas emissions have created a need for an improvement in this process.

A study has been conducted that suggests the energy balance for a mega-methanol plant that produces 1,750M tonnes/Yr of dry MeOH is as follows.

| | M | | | | Energy | Carbon |
|---|---|---|---|---|---|---|
| | tonnes/ | Bbls/ | MM | MMSCF/ | Effi- | Effi- |
| Component | Yr | day | BTU/yr | Yr | ciency | ciency |
| Natural Gas | 973 | | 53,375 | 50,800 | 100 | 100 |
| MeOH | 1,750 | 38,142 | 37,643 | | 71 | 90 |
| LPG | 103 | 3,335 | 4,812 | | | |
| Gasoline | 658 | 15,813 | 29,018 | | | |
| Sum | 761 | 19,148 | 33,830 | | 63 | 89 |

Note
the value for a Mega Methanol plant is 1,750 M tonnes/yr where the ‘M’ refers to thousands. 1,750 M tonnes/yr = 1,750,000,000 kilograms/yr

Only 37% of the energy in the starting natural gas is in the final gasoline and LPG products. The remainder is lost in the form of energy released during the exothermic processes and in the form of by-product hydrocarbons and CO₂. 29% of the energy is lost in the methanol synthesis step and an additional 8 percent is lost during the MTG step. When conducted at remote locations, there is often no use for this energy. It is produced in the form of steam and the heat content of this steam is lost to the environment. Similarly, at refineries where MTG and MTO processes are practices, utilization of energy produced from these processes can also be improved.

### SUMMARY

A process is disclosed for the conversion of a hydrocarbonaceous assets into gasoline. The hydrocarbonaceous assets are converted at a first methanol production facility into methanol by processes comprising gasification and methanol synthesis. At least a portion of the methanol is shipped to a second gasoline production facility that is at least 10 miles in distance from the first location. At the second gasoline production facility, at least a portion of the methanol is converted by a MTG process to produce a gasoline and LPG components and steam. At least a portion of the methanol may also be used in a methanol to olefin process to produce olefins and steam. The olefins may be alkylated with olefins to produce alkylates which may mixed with the gasoline. The steam from one or both of the MTG or MTO processes may also be used by processes such as driving pumps to unload the methanol, driving pumps to deliver the gasoline product, driving compressors to liquefy the LPG components, driving generators to produce electricity, heating other substances, to gasifying imported LNG, and combinations thereof.

Energy management is improved and greenhouse gases are reduced by converting the hydrocarbonaceous asset into methanol, shipping the methanol to a second site near the markets, and converting the methanol into gasoline and producing by-product heat. The by-product heat is used in the methanol handling, methanol conversion process or in other useful ways thus reducing giving the desired improvements in overall energy management and greenhouse gas emissions.

In one embodiment, a method is provided for combining MTG and MTO processes to improve the yield of methanol that can be converted into gasoline.

In another embodiment, an improved use is provided for propylene, and butene by-products from MTO operations producing ethylene.

In another embodiment, the heat produced from one or both of the MTG and/or MTO processes can be used to produce steam which can be used in a variety of processes at the gasoline production facility.

### BRIEF DESCRIPTION OF THE DRAWINGS

These and other objects, features and advantages of the present invention will become better understood with regard to the following description, pending claims and accompanying drawings where:

FIG. 1 shows a block diagram of one exemplary embodiment of a process wherein a hydrocarbonaceous asset is converted to methanol at a methanol production facility, transported to a distant gasoline production facility, and a first portion of the methanol is converted using a methanol to gasoline (MTG) process to produce a first gasoline stock and isoparaffins and a second portion of the methanol is converted in a methanol to olefin (MTO) process to produce olefins and then the isoparaffins and olefins are alkylated into alkylate that can be blended with the first gasoline stock to make a second gasoline stock; and

FIG. 2 shows a second block diagram showing that as part of the MTG and MTO processes, steam is produced which can be utilized, by way of non-limiting examples, as process heat, to produce electricity, to compress and liquefy LPG, to pump methanol being delivered to the gasoline production facility, to pump gasoline produced by the gasoline production facility, as a source for heating refinery components and to gasify imported LNG.

### DETAILED DESCRIPTION

### Definitions

For the purposes of this patent application, the following definitions shall apply:

Hydrocarbonaceous asset: Heavy petroleum having an API gravity of 10° or less, viscous petroleum having a viscosity of 25 cSt at 100° C. or higher, coal, natural gas, ethane, propane, butanes, pentanes, petroleum products having an API gravity of 10° or less or a viscosity of 25 cSt at 100° C. or higher, tires, municipal waste, oil shale, shale oil, agricultural wastes, wood, algae, waste plastics and combinations.

Production of methanol at a remote location and shipping the methanol to the developed location provides the opportunity to do the MTG step at a developed location where the energy of the MTG step can be utilized. This energy can be used as process heat, to generate electricity, to drive pumps and combinations. Example of how this energy can be used in the MTG process, to power pumps to unload the methanol from the ship which delivered it to port, to power pumps to deliver the gasoline product, to compress and liquefy the LPG components, in the production of electricity for use in the plant or for sale, to heat other substances such as hydrocarbon streams in a refinery, to gasify imported LNG, and combinations. By use of the energy from the MTG process in this fashion, less fuel needs to be burned thus overall useful energy management is improved and greenhouse gas emissions are reduced.

One of the greatest energy needs in the MTG process is the energy needed to compress and liquefy the LNG components (Propane and Butane). These must be compressed, chilled and then expanded. When expanded, a portion of the LNG component is liquefied and the portion which is not liquefied is recycled to the compressor. The compression of the LNG components requires significant energy which can be provided by the steam from the MTG process which is used to drive the compressors.

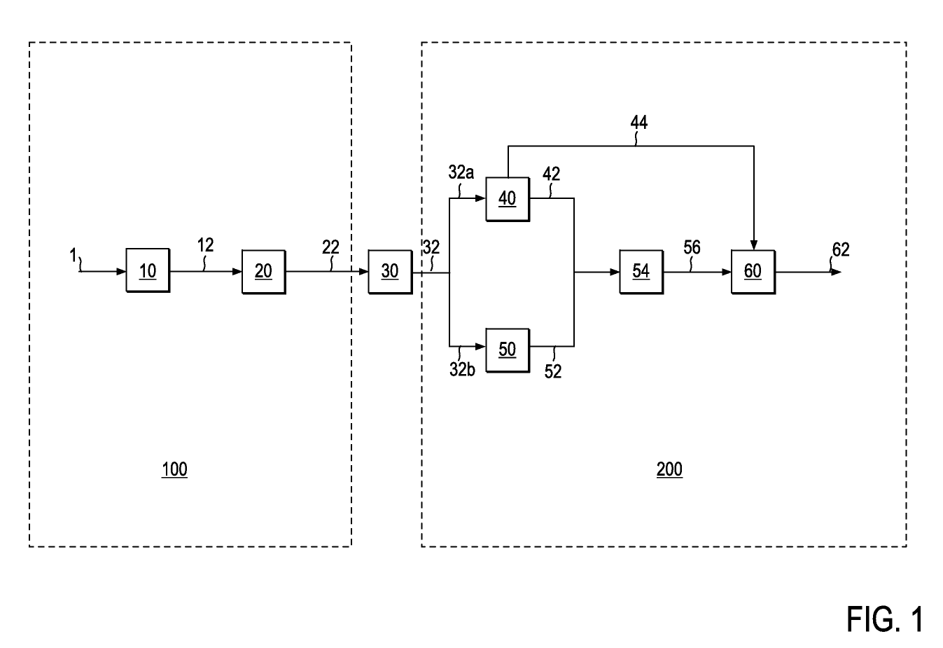

Referring to a first embodiment as illustrated in FIG. 1, a hydrocarbonaceous asset is processed at a methanol production facility such that one of the products is methanol (CH₃OH). The methanol is transported to a distant gasoline manufacturing facility to be further processed into valuable gasoline products.

A first portion of the methanol is converted using a methanol to gasoline (MTO) process into a first gasoline blend stock and isoparaffins. A second portion of the methanol is converted in a methanol to olefin process (MTO) to produce light olefin. The isoparaffins and light olefins are then alkylated to produce alkylates. The first gasoline blend stock is then blended with alkylates to produce a second gasoline blend stock.

### A. Methanol Production from Hydrocarbonaceous Assets

A hydrocarbonaceous asset is converted synthesis gas (syngas) at a methanol production facility, then into methanol, and the methanol can be shipped to a gasoline production facility remote from the methanol production facility. In one embodiment, the distance between the methanol production facility and the gasoline production facility would be 10 or more miles. In another embodiment, the distance would be 100 miles or more. In another embodiment, the distance would be 1000 miles or more.

Syngas can be generated by a wide variety of syngas generation processes. The syngas generator can be a light hydrocarbon reformer or a heavy hydrocarbon reformer. Reforming includes a variety of technologies such as steam reforming, partial oxidation, dry reforming, series reforming, convective reforming, and autothermal reforming. All have in common the production of syngas from methane and an oxidant (steam, oxygen, carbon dioxide, air, enriched air or combinations). The gas product typically contains some carbon dioxide and steam in addition to syngas. Series reforming, convective reforming and autothermal reforming incorporate more than one syngas-forming reaction in order to better utilize the heat of reaction. The processes for producing synthesis gas from C₁-C.sub.3 alkanes are well known to the art. Steam reformation is typically effected by contacting C₁-C₃ alkanes with steam, preferably in the presence of a reforming catalyst, at a temperature of about 1300° F. (705° C.) to about 1675° F. (913° C.) and pressures from about 10 psia (0.7 bars) to about 500 psia (34 bars). Suitable reforming catalysts which can be used include, for example, nickel, palladium, nickel-palladium alloys, and the like. Regardless of the system used to produce syngas it is desirable to remove any sulfur compounds, e.g., hydrogen sulfide and mercaptans, contained in the C₁-C₃ alkane feed. This can be effected by passing the C₁-C₃ alkane gas through a packed bed sulfur scrubber containing zinc oxide bed or another slightly basic packing material. If the amount of C₁-C₃ alkanes exceeds the capacity of the synthesis gas unit, the surplus C₁-C₃ alkanes can be used to provide energy throughout the facility. For example, excess C₁-C₃ alkanes may be burned in a steam boiler to provide the steam used in a thermal cracking step. Examples of the use of light hydrocarbon reformers to make synthesis gas are shown by Baade, Pareky and Venkat in Kirk-Othmer Encyclopedia of Chemical Technology Published Online (2002), Vol 13, Hydrogen, p. 773-784.

In a heavy hydrocarbon reformer, the process involves converting coal, heavy petroleum stocks such as resid, or combinations thereof, into syngas. The temperature in the reaction zone of the syngas generator is about 1800° F.-3000° F. and the pressure is about 1 to 250 atmospheres. The atomic ratio of free oxygen in the oxidant to carbon in the feedstock (O/C, atom/atom) is about 0.6 to 1.5, preferably about 0.80 to 1.3. The free oxygen-containing gas or oxidant may be air, oxygen-enriched air, i.e., greater than 21 up to 95 mole % O₂ or substantially pure oxygen, i.e., greater than 95 mole % O₂. The effluent gas stream leaving the partial oxidation gas generator generally has the following composition in mole % depending on the amount and composition of the feed streams: H₂: 8.0 to 60.0; CO: 8.0 to 70.0; CO₂: 1.0 to 50.0, H₂O: 2.0 to 75.0, CH₄: 0.0 to 30.0, H₂S: 0.1 to 2.0, COS: 0.05 to 1.0, N₂ 0.0 to 80.0, Ar: 0.0 to 2.0. Particulate matter entrained in the effluent gas stream may comprise generally about 0.5 to 30 wt. % more, particularly about 1 to 10 wt. % of particulate carbon (basis weight of carbon in the feed to the gas generator). Fly ash particulate matter may be present along with the particulate carbon and molten slag. Conventional gas cleaning and/or purification steps may be employed such as that described in U.S. Pat. No. 5,423,894. Examples of a heavy hydrocarbon reformer used on coal are by Shadle, Berry and Syamla in Kirk-Othmer Encyclopedia of Chemical Technology Published Online (2002), Vol 6 Coal Gasification p. 771-832. The above Kirk-Othmer reference to Hydrogen also provides other examples of heavy hydrocarbon reformers.

Methanol can be manufactured from a number of hydrocarbonaceous sources such as that described in U.S. Pat. No. 3,898,057, which is hereby incorporated by reference in its entirety. Natural gas is converted into syngas, i.e., carbon monoxide and hydrogen gas mixture, and then catalytically converted into methanol. U.S. Pat. No. 4,407,973, describes a process which uses the methanol synthesis gas from steam reforming in a first methanol plant and effectively integrates a second methanol plant which uses as the methanol synthesis gas (a) the purge gas from the first methanol plant and (b) the clean syn-gas produced by partial oxidation. U.S. Pat. No. 6,645,442, entitled, Method and Apparatus for Producing Methanol making use of Biomass Material, is yet another manner in which a carbonaceous material may be converted to produce methanol. These patents are also incorporated by reference in their entireties. Example of processes and conditions to manufacture methanol are described by English, Brown, Rovner, and Daves in Kirk-Othmer Encyclopedia of Chemical Technology Published Online (2005), Vol 16, Methanol, p 299-316.

U.S. Pat. No. 6,632,971, hereby incorporated by reference in its entirety, describes the potential manufacture of methanol from a source of natural gas. A synthesis gas is used as an intermediate. The synthesis gas can be generated using steam methane reforming, partial oxidation or gasification, or a combined reforming or authothermal reforming process.

Most commercial methanol synthesis plants operate in a pressure range of about 700-2000 psig using various copper based catalyst systems depending on the technology used. A number of different state-of-the-art technologies are known for synthesizing methanol, and are commonly referred to as the ICI (Imperial Chemical Industries) process, the Lurgi process, and the Mitsubishi process.

The methanol syngas, also referred to as “stoichiometric ratioed synthesis gas”, from the syngas generation unit is fed to a methanol synthesis reactor at the desired pressure of about 700 to 2000 psig, depending upon the process employed. The syngas then reacts with a copper based catalyst to form methanol. The reaction is exothermic. Therefore, heat removal is ordinarily required. The raw or impure methanol is then condensed and purified to remove impurities such as higher alcohols including ethanol, propanol, and the like. The uncondensed vapor phase comprising unreacted methanol syngas is recycled to the feed.

The operation of compressing the methanol synthesis gas requires expensive equipment that is costly to maintain. Moreover, the need to compress the methanol synthesis gas to reach suitable operating pressures for the methanol synthesis operation further increases the production cost of methanol. For optimal methanol production, U.S. Pat. No. 5,496,859 teaches using a stoichiometric ratioed syngas supplied to the methanol synthesis unit generally conforming to the following specifications: (H₂−CO₂)/(CO+CO₂)=1.9-2.1, and N₂, Ar and CH₄.≦3.0% and H₂O. This process partially oxidizes natural gas in a gasifier to produce hot pressurized syngas which is passed through a steam reforming catalytic reactor to produce a reformer syngas, a portion of which is recycled as feed to the gasifier while the remaining portion is combined with partially cooled gasifier syngas exiting the catalytic reactor to form a stoichiometric ratioed syngas. The ratio adjusted syngas then enters a methanol synthesis unit at conditions necessary to convert it to methanol with little or no external compression.

### B. Transportation of Methanol from Methanol Production Facility to a Gasoline Manufacturing Facility

Methanol produced at the methanol production facility may be transported to the gasoline manufacturing facility in a variety of manners. For example, floating carrier vessels or tankers may be used to transport the methanol across large bodies of water such as oceans or seas. Alternatively, pipelines could be used to move the methanol at least partially between methanol and gasoline production facilities. Trains having tanker cars may be used to partially, convey the methanol. Of course, trucks may also be used to transport the methanol in certain cases.

### C. MTG Production of First Gasoline Stock and Isoparaffins

As seen in FIG. 1, the methanol (32) is received at the gasoline manufacturing facility (200) remote from the methanol production facility (100). The stream of methanol (32) is split into a first portion (32a) and a second portion (32b). Conventional methanol to gasoline (MTG) processes (40) may then be used to convert the first portion of methanol into a first gasoline blend stock (44) and into non-quaternary isoparaffins (42). Non-quaternary isoparaffins are produced consisting of isobutane, isopentane, 2-methylpentane, 3 methylpentane, 2,3-dimethylbutane and combinations. The MTG process will also produce normal paraffins consisting of n-butane, n-pentane, n-hexane and combinations. Optionally these normal paraffins can be isomerized to form additional non-quaternary isoparaffins and used in the alkylation process.

U.S. Pat. No. 6,046,372 incorporated herein in its entirety by reference, provides many examples using modified medium pore zeolite catalysts, e.g., a shape-selective crystalline silicate catalyst selected from the group consisting of ZSM-5, ZSM-11, ZSM-12, ZSM-23, ZSM-35, ZSM-48, and MCM-22, to produce ethylene, propylene, p-xylene, and gasoline precursors from methanol at commercially attractive partial pressures between 15 and 170 psia. The reference teaches that ethylene+propylene selectivity is optimized by using between 1 and 20 wt % toluene co-feed, ZSM-5 catalysts with d/r² values between 0.5 and 20, and temperatures between 380° and 500° C.

U.S. Pat. No. 5,248,647 incorporated herein by reference, describes the use of SAPO-34 type catalysts for the conversion of methanol or dimethyl ether to C.2 C₅ olefins at commercially attractive conversions of methanol exceeding 98%. The patent teaches that ethylene+propylene selectivity is optimized at temperatures between 400° and 500° C. and methanol pressures between 5 and 40 psia. The '372 and '647 referenced methanol conversion methods are especially suited to use in the present invention.

Preferably, the present invention can employ an olefin production zone containing a metal aluminophosphate catalyst selected from the group consisting of SAPO-34, SAPO-17, SAPO-18, and mixtures thereof, the catalyst being described in U.S. Pat. Nos. 4,440,871, 5,126,308, and 5,191,141 and hereby incorporated by reference.

U.S. Pat. No. 3,928,483 incorporated herein in its entirety by reference, describes the use of shape-selective zeolites such as ZSM-5 for the conversion of methanol or dimethyl ether to gasoline. U.S. Pat. Nos. 3,911,041, 4,025,571, 4,025,575, and 4,052,479 describe the use of shape-selective zeolites in converting methanol and/or dimethyl ether to olefins, to aromatic hydrocarbons, or to mixtures thereof. The foregoing patents are incorporated herein by reference as background material.

U.S. Pat. No. 4,499,314 incorporated herein by reference, discloses that the addition of various promoters, including aromatic compounds, such as toluene, accelerate the conversion of methanol to hydrocarbons over zeolites, such as ZSM-5, which have a pore size sufficient to permit sorption and diffusion of the promoter. In particular, the '314 patent teaches that the increased conversion resulting from the addition of the promoter allows the use of lower severity conditions, particularly lower temperatures, which increase the yield of lower olefins (column 4, lines 17-22). Thus in Example 1 of the patent the addition of toluene as a promoter reduces the temperature required to achieve full methanol conversion from 295° C. to 288° C. while increasing the ethylene yield from 11 wt % to 18 wt %. In the Examples of the '314 patent the methanol feedstock is diluted with water and nitrogen such that the methanol partial pressure is less than 2 psia.

### D. MTO Production of Olefins

The second portion of methanol (32b) is converted in a methanol to olefin process (50) to produce light olefins (52) consisting of ethylene, propylene, butylenes and combinations.

U.S. Pat. No. 4,677,242 (Kaiser) incorporated herein in its entirety by reference, describes the use of a silicoaluminophosphate (SAPO) molecular sieve catalyst for converting various oxygenates, such as methanol, to olefins. According to the patent, the SAPO catalyst is an extremely efficient catalyst for the conversion of oxygenates to prime olefin products when the feed is converted in the presence of a diluent. The diluent used has an average kinetic diameter larger than the pores of the SAPO molecular sieve. The selected SAPO molecular sieves have pores that an average kinetic diameter characterized such that the adsorption capacity (as measured by the standard McBain-Bakr gravimetric adsorption method using given adsorbate molecules) shows adsorption of oxygen (average kinetic diameter of about 3.36 angstroms) and negligible adsorption of isobutane (average kinetic diameter of about 5.0 angstroms).

U.S. Pat. No. 6,316,683, incorporated herein in its entirety by reference, describes a method for making an olefin product from an oxygenate feedstock while protecting the catalytic activity of a silicoaluminophosphate molecular sieve used for catalyzing the reaction. Prior to use, the molecular sieve is protected by shielding with a template molecule or by carbonaceous material on the surface of the molecular sieve material. After removing the template or carbonaceous material to activate the molecular sieve, catalytic activity is protected by maintaining the temperature of the molecular sieve above 150° C. Alternatively, the activated catalyst can be exposed to temperatures below 150° C. by preventing exposure of catalyst active sites to water. U.S. Pat. No. 6,166,282, incorporated herein in its entirety by reference, describes a method for making an olefin product from an oxygenate feedstock. The oxygenate feedstock is exposed to a catalyst bed that facilitates the reaction. During the reaction, a carbonaceous product builds up on the catalyst particles. The catalyst particles are passed through a regenerator to remove the carbonaceous product.

### E. Alkylation of Isoparaffins and Olefins

At least a portion of these light olefins (52) are alkylated with the light non-quaternary isoparaffins (42) produced in the MTG process to produce alkylates (52). Preferably the light olefin is ethylene, and the alkylation process uses an ionic liquid catalyst. At least a portion of the non-quaternary isoparaffins are alkylated with at least a portion of the light olefins to produce alkylate. Preferably, the light olefins contain ethylene in an amount greater than 50 wt % and the alkylation is done with an ionic liquid catalyst.

U.S. Patent No. 7,432,408 (Timken et al.), hereby incorporated by reference in its entirety, teaches the alkylation of isoparaffins and olefins. In the present exemplary embodiment, the olefins are received as methanol to olefin unit offgas. The preferred olefin is ethylene. This stream may also contain propylene, butylenes and pentenes. The offgas is preferably passed through an ethylene extraction unit to produce a C₂+ fraction, which is rich in ethylene, typically about 50 vol %, and a lighter fraction, which is rich in hydrogen. The C₂+ fraction is fed to an alkylation reactor.

The isoparaffins preferably include isopentane. The isopentane-containing stream may also contain other isoparaffins such as isobutane. A large number of liquid or solid acid catalysts are known which are capable of effecting alkylation of isoparaffins such as isobutane or isopentane by olefins such as propylene, 1-butene, 2-butene and isobutylene. The catalysts which are most widely used in industrial practice are concentrated sulfuric acid and hydrofluoric acid alone or mixed with Lewis acids such as boron trifluoride.

Those processes suffer from major disadvantages: hydrofluoric acid by virtue of its toxicity and its high degree of volatility; and sulfuric acid by virtue of a substantial volumetric consumption of the catalyst requiring burdensome regeneration. These reasons have motivated the development of catalysts which are solid or which are supported on solids such as aluminosilicates or metal oxides such as zirconia treated with sulfuric acid. However, solid catalysts are generally found to present a low level of selectivity and a low degree of activity. The use of aluminum chloride has also been studied and proposed.

The process according to the present embodiment preferably employs a catalytic composition comprising at least one aluminum halide and at least one quaternary ammonium halide and/or at least one amine halohydrate. The aluminum halide which can be used in accordance with the invention is most preferably aluminum chloride.

The quaternary ammonium halides which can be used in accordance with the invention are those described in U.S. Pat. No. 5,750,455, which is incorporated by reference herein, which also teaches a method for the preparation of the catalyst.

The ionic liquid catalysts which are most preferred for the process of the present invention are N-butylpyridinium chloroaluminate (C₅H₅C₄H.sub.9Al₂Cl₇). A metal halide may be employed as a co-catalyst to modify the catalyst activity and selectivity. Commonly used halides for such purposes include NaCl, LiCl, KCl, BeCl₂, CaCl₂, BaCl₂, SiCl₂, MgCl₂, PbCl₂, CuCl, ZrCl₄, AgCl, and PbCl₂ as published by Roebuck and Evering (Ind. Eng. Chem. Prod. Res. Develop., Vol. 9, 77, 1970). Preferred metal halides are CuCl, AgCl, PbCl₂, LiCl, and ZrCl₄.

HCl or any Broensted acid may be employed as an effective co-catalyst. The use of such co-catalysts and ionic liquid catalysts that are useful in practicing the present invention is disclosed in U.S. Published Patent Application Nos. 2003/0060359 and 2004/0077914. Other co-catalysts that may be used to enhance the catalytic activity of ionic liquid catalyst system include IVB metal compounds preferably metal halides such as TiCl₃, TiCl₄, TiBR₃, TiBR₄, ZrCl₄, ZrBr₄, HfCL₄, HfBr₄, as described by Hirschauer et al. in U.S. Pat. No. 6,028,024.

It is especially important to note that H₂SO₄ and HF are not effective for the alkylation of isoparaffins with ethylene. Therefore, if alkylation 54 is used with ethylene, the aforementioned ionic liquid catalyst should be used rather than using an alkylation process utilizing H₂SO₄ and HF.

### F. Reaction Conditions

Due to the low solubility of hydrocarbons in ionic liquids, olefins-isoparaffins alkylation, like most reactions in ionic liquids is generally biphasic and takes place at the interface in the liquid state. The catalytic alkylation reaction is generally carried out in a liquid hydrocarbon phase, in a batch system, a semi-batch system or a continuous system using one reaction stage as is usual for aliphatic alkylation. The isoparaffin and olefin can be introduced separately or as a mixture. The molar ratio between the isoparaffin and the olefin is in the range 1 to 100, for example, advantageously in the range 2 to 50, preferably in the range 2 to 20. In a semi-batch system the isoparaffin is introduced first then the olefin, or a mixture of isoparaffin and olefin. Catalyst volume in the reactor is in the range of 2 vol % to 70 vol %, preferably in the range of 5 vol % to 50 vol %. Vigorous stirring is desirable to ensure good contact between the reactants and the catalyst. The reaction temperature can be in the range −40° C. to +150° C., preferably in the range −20° C. to +100° C. The pressure can be in the range from atmospheric pressure to 8000 kPa, preferably sufficient to keep the reactants in the liquid phase. Residence time of reactants in the vessel is in the range a few seconds to hours, preferably 0.5 min to 60 min. The heat generated by the reaction can be eliminated using any of the means known to the skilled person. At the reactor outlet, the hydrocarbon phase is separated from the ionic phase by decanting, then the hydrocarbons are separated by distillation and the starting isoparaffin which has not been converted is recycled to the reactor.

Typical reaction conditions may include a catalyst volume in the reactor of 5 vol % to 50 vol %, a temperature of −10° C. to 100° C., a pressure of 300 kPa to 2500 kPa, an isoparaffin to olefin molar ratio of 2 to 8 and a residence time of 1 min to 1 hour.

A catalyst system comprised of aluminum chloride and hydrogen chloride (hydrochloric acid) for catalyzing the alkylation of iso-paraffins and olefins in ionic liquids (chloroaluminate ionic liquids) is preferred. The HCl can be used as a co-catalyst to enhance the reaction rate. For example, the alkylation of isopentane with ethylene in a batch autoclave is complete in <10 minutes in the presence of HCl. In the absence of HCl, the reaction usually takes ½ hour to 1 hour time (50° C. and autogenic pressure of about 965 kPa and feed ratio of about 4). The product selectivity was comparable to that of chloroaluminate ionic liquid without the presence of HCl.

### G. Exemplary Embodiment

A scheme for an integrated refinery alkylation process to implement an embodiment of the present embodiment is shown in FIG. 1

At a methanol production facility 100a hydrocarbonaceous asset 1 is converted in a syngas generator 10 to syngas 12. The syngas is converted in a methanol synthesis plant 20 to methanol 22. The methanol is shipped 30 to a gasoline production facility 200 which is 100 miles or more from the methanol production facility. The methanol received at the gasoline manufacturing facility 32 is split into two streams 32a and 32b. Stream 32b is processed in a MTO plant 50 to make light olefins 52. Stream 32a is processed in a MTG plant 40 to make non-quaternary isoparaffins 42 and a first stream of gasoline blend stock 44. The non-quaternary isoparaffins and the light olefins are fed to an alkylation plant 50 to product alkylate 52. The first streams of gasoline blend stock and alkylate blended in a blend plant 50 to produce a second stream of gasoline blend stock 62.

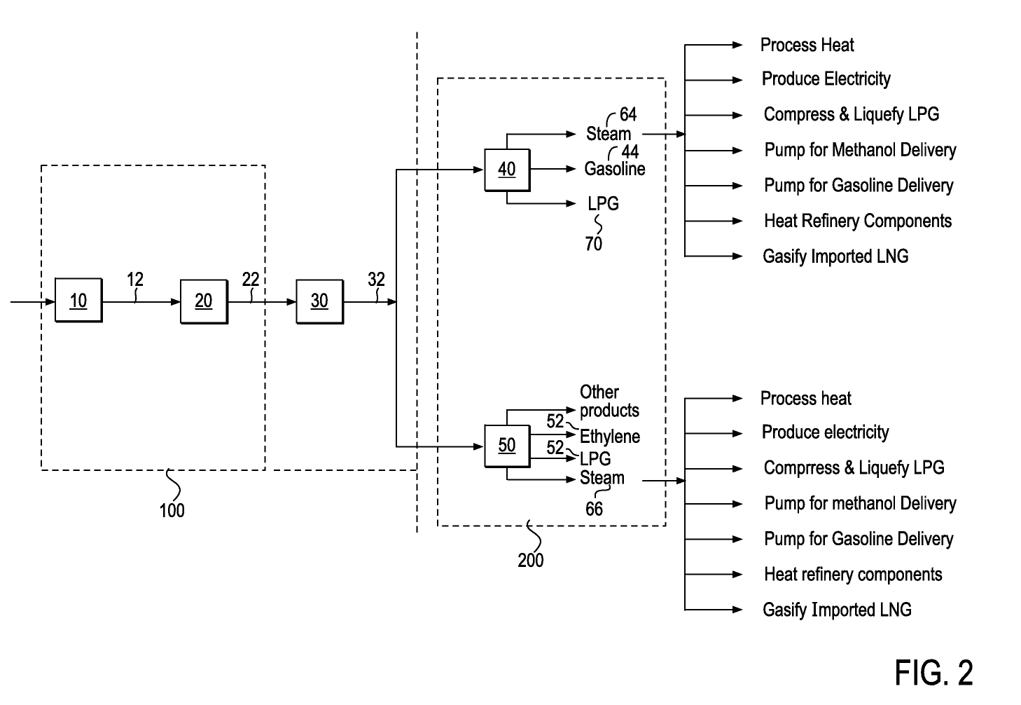

Referring now to FIG. 2, heat from the MTG and MTO processes can be converted to steam to provide energy for second production facility 200 needs. As MTG and MTO processes generally generate a lot of heat, steam produced from these processes can be used to improve the operating efficiency of facility 200. For example, steam 64 and 66 can be used as a source of process heat. Alternatively, steam 64 and 66 could drive turbines to produce electricity. Also, LPG 70 may be produced as a part of the MTG process. In such a case, the steam could be used to drive generators and pumps to compress and liquefy the LPG. Also, steam 64 and 66 could be use to pump methanol being delivered to facility 200. The steam could also be employed to pump gasoline produced by facility 200. If LNG is available to facility 200, the steam could act as a source to gasify imported LNG.

Another potential use for the steam is to meet the energy needs of the alkylation unit. In particular, energy provided by the steam may be used in distillation and pumping of recycled isoparaffins.

Alkylation reactions in accordance with the present embodiment may be conducted in one or more alkylation zone using the same or different ionic liquid catalysts. For example, the C₂₊ fraction described above may contain propylene, butylene and/or pentenes and the isopentane containing stream may also contain isobutane. Isobutane may be alkylated with ethylene to produce a high-octane C₆ gasoline blending component. A C₄ olefin containing stream may be isolated and used for the alkylation of isobutane, isopentane or their mixtures. Other variations and combinations will be apparent to refiners generally.

While in the foregoing specification this invention has been described in relation to certain preferred embodiments thereof, and many details have been set forth for purpose of illustration, it will be apparent to those skilled in the art that the invention is susceptible to alteration and that certain other details described herein can vary considerably without departing from the basic principles of the invention.

For example, the methanol can be produced at more than one methanol production facility. Also, equipment at the methanol production facility can be moved to a new location when the hydrocarbonaceous asset is exhausted. Non-quaternary isoparaffins and normal from the refining of petroleum or synthetic crudes can also be blended with the non-quaternary isoparaffins derived from methanol via the MTG process. Olefins from the refining of petroleum or synthetic crudes, or from dehydrogenation of light paraffins can also be blended with the olefins derived from methanol via the MTO process. The alkylation process can consist of independent steps which may use different processes, catalysts and conditions. For example, the ethylene can be alkylated with isopentane using an ionic liquid catalyst, and the propylene and butenes alkylated with isobutane using sulfuric acid. Other combinations are possible and are within the spirit of the invention. If ethylene is not to be alkylated, then conventional alkylation unit using HF and H₂SO₄ may be used in place of the liquid ionic catalysts.

What is claimed is:

1. The conversion of a hydrocarbonaceous asset into gasoline comprising:
converting the hydrocarbonaceous assets at a first methanol production facility into methanol by processes comprising gasification and methanol synthesis;
shipping at least a portion of the methanol to a second gasoline production facility which is at least 10 miles in distance from the first methanol production facility;
converting at the second gasoline production facility at least a portion of the methanol by a MTG process to produce a gasoline, LPG components and steam; and
using the steam by processes selected from at least one of driving pumps to unload the methanol, driving pumps to deliver the gasoline product, driving compressors to liquefy the LPG components, driving generators to produce electricity, heating other substances, to gasifying imported LNG, and combinations thereof.

2. The method of claim 1 further comprising:
converting at the second gasoline production facility at least a portion of the methanol by an MTO process to at least produce olefins and steam.

3. The method of claim 2 wherein:
the steam and energy produced in one of the MTG and MTO processes is used in an alkylation step to alkylate olefins.

4. The method of claim 3 wherein:
the olefin is ethylene.

5. A method of converting methanol into gasoline, the method comprising the steps of:
(a) receiving a stream of methanol at a gasoline production facility;
(b) splitting the stream into a first stream for use in a methanol to gasoline (MTG) facility and a second stream for use in a methanol to olefin (MTO) facility;
(c) converting the first stream of methanol using a methanol to gasoline process to produce a first stream of gasoline blend stock and a stream of light non-quaternary isoparaffins and steam;
(d) converting the second stream of methanol using a methanol to olefin process to produce a stream of light olefins selected from the group consisting of ethylene, propylene, butenes and combinations thereof and steam;
(e) alkylating at least a portion of the stream of light non-quaternary isoparaffins with at least a portion of the stream of light olefins to produce an alkylate and steam;
(f) blending at least a portion of the alkylate with at least a portion of the first stream gasoline blend stock to form a second stream of gasoline blend stock; and
(g) using the steam by processes selected from the group consisting of driving pumps to unload the methanol, driving pumps to deliver the gasoline product, driving compressors to liquefy the LPG components, driving generators to produce electricity, heating other substances, to gasifying imported LNG, and combinations.

6. The process according to claim 5:
wherein the alkylation process uses an ionic liquid catalyst.

7. The process of claim 6 wherein:
the alkylation process uses ethylene.

8. The process according to claim 5 further comprising:
the manufacture of the methanol stream received at the gasoline production facility at a methanol manufacturing facility where the distance between the gasoline manufacturing facility and the methanol manufacturing facility is 10 miles or more.

9. The process according to claim 6 wherein:
the distance is 100 miles or more.

10. The process according to claim 7 wherein:
the distance is 1000 miles or more.
